(19) United States
(12) Patent Application Publication
(10) Pub. No.: US 2021/0205782 A1
(43) Pub. Date: Jul. 8, 2021

(54) MULTILAYER ABSORBENT FILTERING ITEM COMPRISING A FIRST LAYER OF A POROUS ACTIVATED CARBON SUPPORT DISPOSED ON A NONWOVEN POLYESTER-FIBRE FELT, A SECOND LAYER OF A POLYMER FILM AND A THIRD LAYER OF AN ACTIVE INGREDIENT; METHOD FOR OBTAINING SAID ITEM; AND USE OF SAME

(71) Applicant: COMERCIALIZADORA INNVENTO S.A., Vina del Mar (CL)

(72) Inventor: Igor Francisco Araya Lazo, Vina del Mar (CL)

(21) Appl. No.: 17/254,894

(22) PCT Filed: Jun. 22, 2018
(86) PCT No.: PCT/CL2018/000022

§ 371 Date: Dec. 22, 2020

(51) Int. Cl. B01J 20/28; B01J 20/20; B01J 20/06; B01J 20/26; B01J 20/32; A61L 9/014; A01K 31/18; A01K 1/015

(52) U.S. Cl. CPC B01J 20/28035; A61L 2209/14; B01J 20/06; B01J 20/262; B01J 20/28028; B01J 20/28064; B01J 20/28071; B01J 20/3212; B01J 20/3295; B01J 20/3289; A61L 9/014; A01K 31/18; A01K 1/015; A61L 2209/22; B01J 20/20

(57) ABSTRACT

The present invention relates to a multilayer absorbent filtering item comprising: a first layer of a porous activated carbon support disposed on a nonwoven polyester-fibre felt; a second layer, where the surface of the first layer is bonded to a film of polymer and low-foam surfactants (mixture A); and a third layer, where the surface of the second layer is bonded to a film comprising an active ingredient and a low-foam surfactant in an acid aqueous medium (mixture B), the top face of said polymer film being bonded to the active ingredient, and the bottom face thereof adhering to the activated carbon, by means of polar and non-polar interactions. Also disclosed are a method for obtaining the multilayer absorbent filtering item and the use of the multilayer absorbent filtering item.

The present invention concerns the development of an innovative technology directed to a multilayer filter-adsorbent article to eliminate offensive odors from the breeding of poultry animals, such as swine and birds.

Specifically, a multi-layer filter-adsorbent article is disclosed that fulfills the function of continuously degrading the compounds responsible for the offensive odors, meaning the odor generated by substances or industrial, commercial or service activities that produce nuisance, even if it does not cause harm to human health, such as those generated in an animal farm due to the presence of volatile organic compounds (VOCs) (ammonia, hydrogen sulfide, methane and carbon dioxide), which are the product of the decomposition of organic waste from animals. A process for obtaining such multilayer filter-adsorbent article is also disclosed.

DESCRIPTION OF WHAT IS KNOWN IN THE FIELD

It is known that porous materials have covered numerous areas of research due to the applications that can be obtained with them. These materials are used as adsorbents, ion exchange systems, composite separations, and as catalysts or catalytic supports.

Although porous solids have a varied composition, they have in common the accessible space inside their structure. In other words, the aggregation of small particles of the solid results in the formation of pores within these grains and is defined as intra-particles or textural porosity. The diameter of the textural pores is directly related to the size of the grains forming these pores.

In the prior art different types of adsorbent structures and processes of application thereof have been disclosed, specifically document C1200901573 refers to a process for impregnating a porous solid adsorbent, charcoal, with permanganate comprising inerting charcoal with hydrophobic solvent applying vacuum, contacting inerted charcoal with aqueous solution of Na, K or Li permanganate, removing excess liquid and drying resulting solid; and porous solid adsorbent.

On the other hand, the presentation WO 2015/57873 discloses a procedure to impregnate a porous support with chemical agent(s), a porous support and a system to impregnate such support. It aims to describe a procedure to impregnate a porous support with chemical(s), the optimum porous support to be used depending on the surface area and a system specifically designed to carry out the impregnation process.

In addition, document WO1995013122 reveals a process for impregnating zeolite with a quaternary ammonium cation (QAC) and then coating the permanganate impregnated zeolite (such as potassium permanganate) and for impregnating zeolite with permanganate and then coating the QAC impregnated zeolite, and impregnated and coated zeolite crystals resulting from either process. Either coating acts as a protective agent for the impregnation substance within each zeolite crystal and allows controlled release of the impregnation substance, thus allowing a controlled rate of diffusion (or absorption) in applications where coated impregnated zeolite is used to absorb contaminants from the air or water. The combinations of coated and uncoated zeolite crystals can be chosen to match specific environmental circumstances which can be calculated by analysis of the air or water to be treated. Coated and uncoated QAC impregnated zeolite mixtures can be used to react with organic compounds such as benzene, toluene and xylene, uncoated zeolite impregnated with permanganate, and mixtures of coated and uncoated permanganate impregnated zeolite can be used to react with hydrogen sulfide, acetone, ethylene glycols, formaldehyde and other contaminants.

Methods for producing manganese dioxide impregnated zeolite crystals, and for using such manganese dioxide impregnated crystals to absorb contaminants from the fluid, are also reported.

In addition, U.S. Pat. No. 8,664,153 refers to an adsorbent composition comprising: an activated carbon impregnated with at least one saturated long chain aliphatic hydrocarbon, a permanganate salt, and iron (III) oxide. Adsorbent compositions resulting from such processes are also disclosed in this document.

The present application presents an original multilayer filter-absorbent article, with innovative technological improvements, which aims to mitigate the odors from the breeding of poultry animals, such as swine and birds.

This multilayer filter-absorbent article comprises a first layer of a porous activated carbon support arranged on a non-woven polyester fiber felt, where the high surface area of the activated carbon is joined to a film of a polymer (applying mixture A), where its upper side is joined to an active agent and its lower side is attached to the activated carbon by means of polar and non-polar interactions (applying mixture B). Where the polymer is a liquid silicone, such as dimethylpolysiloxane, and the active agent, such as a potassium permanganate salt.

### EXAMPLE OF APPLICATION

For the evaluation of the effectiveness of the odor filter-adsorbent article, source samples were prepared based on NCh 3386 and Dynamic Olfactometry Analysis according to NCh 3190:2010, which allows the measurement of odor concentration and approves the international standard EN 13725:2004.

Sixteen samples were taken where 8 samples were compared without considering a filtering article, 6 samples considering the multilayer filter-adsorbent article of the present invention and two samples with the filtering article in the state prior to the present invention.

An analysis of the intensity of odors (dynamic olfactometry) in the air samples was considered, considering the multilayer filter-adsorbent article or not, as the case may be. It is stated that “pig smell” is CHARACTERISED by a high concentration of ammonia (NH3), hydrogen sulfide (H2S) and other volatile organic compounds (VOCs).

The evaluation was carried out in the olfactometry laboratory of Ecometrika (an internationally certified company) in Santiago. The tests were carried out according to the international protocols in force and using the “European Odor Unit (ouE/m3)” as measurement standard. It is important to point out that the detection threshold of an odor is recorded at 5 ouE/m3.

For this purpose, a total of 16 recirculation air samples were collected from pig houses, testing liquid and solid slurry for their very different odorant load. Liquid slurry represents tests 1 to 4, while solid slurry is collected in tests 5 to 16.

### Odor Measurement Results

Table 1 shows the results obtained from pig farm tests for liquid manure.

| | | | Odor | |
|---|---|---|---|---|
| | Slurry | Filtrating | Concetration | Concentration |
| Test Number | Type | Element | (uoE) | Variation % |
| Test 1 | Liquid | Without | 96.835 | 97.4% |
| | | Filtrating | | |
| | | Art. | | |
| Test 2 | Liquid | With | 2.545 | |
| | | Filtrating | | |
| | | Ads. Art. | | |
| Test 3 | Liquid | Without | 122.004 | 99.2% |
| | | Filtrating | | |
| | | Art. | | |
| Test 4 | Liquid | With | 939 | |
| | | Filtrating | | |
| | | Ads. Art. | | |
| | | | | 98.3% |

According to the test described in i) of intensity of odors, the effect of the filter-adsorbent article has a great impact generating a decrease in the concentration of the odor of 98.3% on average. It was observed that the reduction in European Odor Units reached 121,065 uoE in the best of the cases seen, with and without the filter-adsorbent article of the invention.

The results of the tests on solid slurry with and without filter-adsorbent article of the invention are presented in Table 2.

It was observed that solid slurry has a much less offensive odorant emission than liquid slurry. The result of these tests showed a decrease in odor concentration of 90.3% on average.

| | | | Odor | |
|---|---|---|---|---|
| | Slurry | Filtrating | Concentration | Concentration |
| Test Number | Type | Element | (uoE) | Variation % |
| Test 5 | Solid | Without | 447 | 95.3% |
| | | Filtrating | | |
| | | Art. | | |
| Test 6 | Solid | With | 21 | |
| | | Filtrating | | |
| | | Ads. Art. | | |
| Test 7 | Solid | Without | 489 | 86.9% |
| | | Filtrating | | |
| | | Art. | | |
| Test 8 | Solid | With | 64 | |
| | | Filtrating | | |
| | | Ads. Art. | | |
| Test 9 | Solid | Without | 512 | 90.4% |
| | | Filtrating | | |
| | | Art. | | |
| Test 10 | Solid | With | 49 | |
| | | Filtrating | | |
| | | Ads. Art. | | |
| Test 11 | Solid | Without | 256 | 88.7% |
| | | Filtrating | | |
| | | Art. | | |
| Test 12 | Solid | With | 29 | |
| | | Filtrating | | |
| | | Ads. Art. | | |
| | | | | 90.3% |

It can be observed that there are differences in the efficiency of the total concentration values, between the tests with and without filter-adsorbent article (tests from 1 to 12), where it is possible to obtain an average in the decrease of the total concentration greater than 93%, (see % variation of the concentrations).

Following are the results of tests 13 to 16, samples testing the filtering article prior to the invention (layer of activated carbon without the treatment), where the average value in the decrease of the total concentration of the gases of 42% is observed, far below 93% of efficiency when the surface of activated carbon is modified, according to the present invention.

| | | | Odor | |
|---|---|---|---|---|
| | Slurry | Filtrating | Concetration | Concentration |
| Test Number | Type | Element | (uoE) | Variation % |
| Test 13 | Solid | With Prior | 117 | 49.6% |
| | | Art Filter | | |
| Test 14 | Solid | With | 59 | |
| | | Filtrating | | |
| | | Ads. Art. | | |
| Test 15 | Solid | With Prior | 140 | 35.0% |
| | | Art Filter | | |
| Test 16 | Solid | With | 91 | |
| | | Filtrating | | |
| | | Ads. Art. | | |
| | | | | 42.3% |

It is concluded that the results of these tests show that the multilayer filter-adsorbent article of the present invention solves the problem of offensive odors produced by pig breeding facilities with a greater efficiency in odor adsorption, with an unexpectedly surprising effect in comparison to a traditional porous adsorbent system.

Also, it is surprising the fast action of the filter-adsorbent article on the pig odor compounds, since it requires a very short contact time to achieve its functional action.

To better understand the invention, it will be described on the basis of figures that have only an illustrative character, the scope of the invention not being limited to the dimensions, nor to the quantity of elements illustrated.

### BRIEF DESCRIPTION OF THE FIGURES

FIG. 1: shows the pavilion where the multilayer filter-adsorbent article is tested.

FIG. 2: shows the pore of a porous activated carbon support prior to the invention (layer 1).

FIG. 3: shows part of the layers of the multilayer filter-adsorbent article of the present invention with the silicone film (layer 2).

FIG. 4: shows the absorbent article of the present invention with the permanganate salt on silicone film (layer 3).

FIG. 5: shows the molecular lamination procedure for obtaining the multilayer filter-adsorbent article used in the application example.

### DETAILED DESCRIPTION OF THE INVENTION

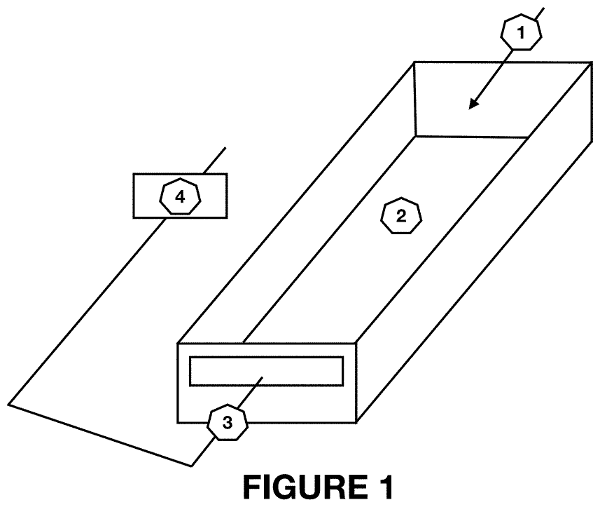

FIG. 1 considers a pavilion with an entrance of ambient air (1), a middle zone that considers the air inside the pavilion (2) and an exit of odorous gases that are channeled through ducts (3), to be treated (filtered).

The odorous gases are captured at the exit of the ducts, either treated considered filter-adsorbent article (4) or untreated, depending on what is required for the tests. Finally, the gases are returned to the environment.

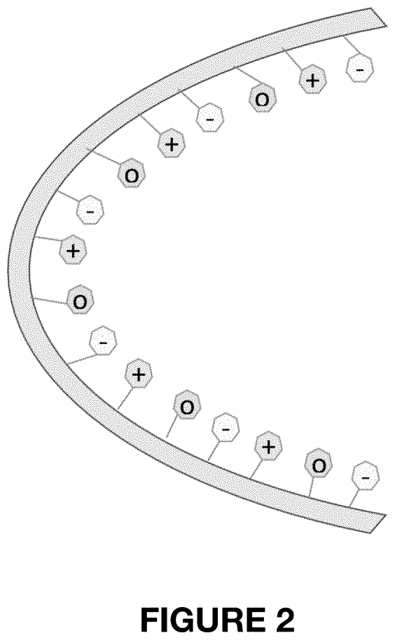

FIG. 2 shows a filter-adsorbent article, which corresponds to layer 1, where the diagram represents the view of a cross section of the surface of the pore of the activated carbon and shows the different zones with polarities and/or positive charges (+), negative charges (−) and non-polar (0).

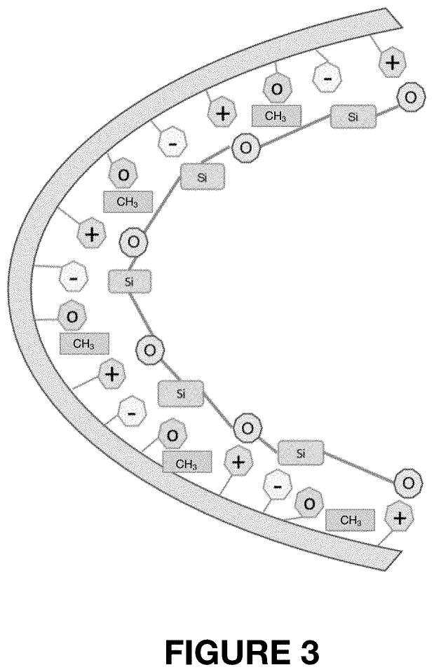

FIG. 3 shows details of the multilayer filter-adsorbent article of the present invention with the silicone film, corresponding to layer 2. Silicone is an inorganic polymer derived from polysiloxane which is made up of a series of alternating silicon and oxygen atoms. It can be observed that the chemical structure of silicon, such as dimethylpolysiloxane, forms a chain starting with a Silica atom (Si) attached, on one side, to two methyl groups (CH3), and on the other end to 1 oxygen atom (O), and so on to form a chain.

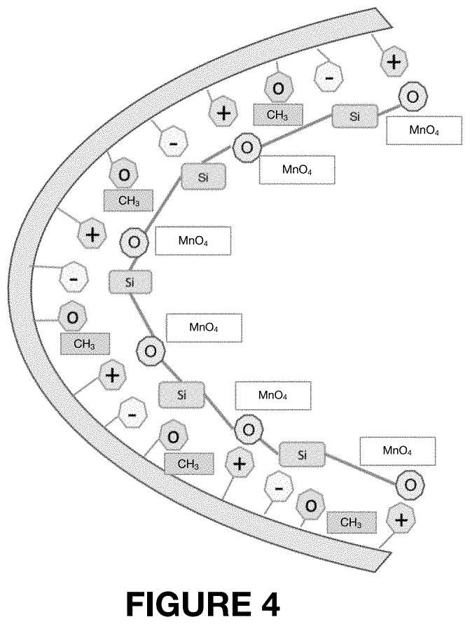

FIG. 4, shows the multilayer filter-absorbent article of the present invention with the permanganate salt on silicone film (corresponding to layer 3). The interaction of the Manganese (Mn) atom with the exposed oxygen (O) atoms of the silicone chain, dimethylpolysiloxane, allows anchoring and thus the third active surface of permanganate salt is formed.

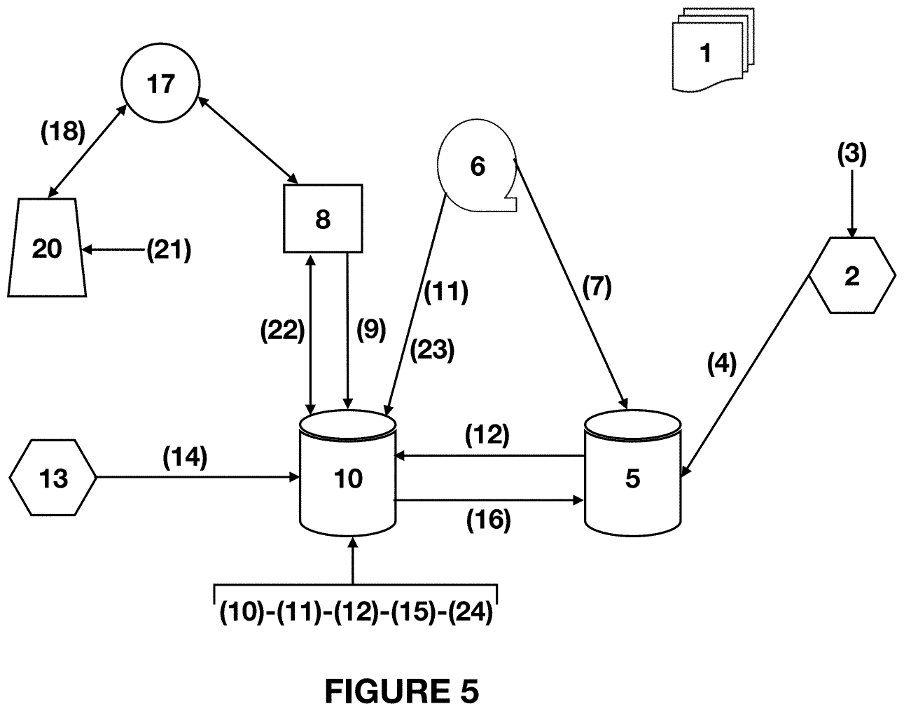

FIG. 5 shows a flow diagram of the system, which together with the flow and process lines show a procedure to achieve a high-performance molecular lamination and to obtain the multilayer filter-adsorbent article, which will be detailed next.

The present invention comprises the multilayer filter-adsorbent article and uses a porous support consisting of a non-woven polyethylene terephthalate (PET) fiber felt impregnated with 40% vegetable activated carbon, having a specific surface area of at least 800 m2/g carbon and a pore volume of at least 0.15 m3/g carbon.

In the state of the art, it has been proposed to solve the problem of non-selective reactivity of the surface of the activated carbon by using mineral oil to cover the surface. This reactivity to be controlled is due to the functional groups such as hydroxyl, carbonyl, carboxyl, among others, which give it the amphoteric character, which can be acidic, positive or basic polarity, negative polarity, (see FIG. 2).

However, mineral oil has several drawbacks; first, it is hydrophobic, so it does not interact with the polar functional groups on the surface of the activated carbon; second, its viscosity and high surface tension prevents it from entering the pore and only manages to cover its external surface, eliminating the main property of the porous support, which is its large available surface area; and finally, the ecological problem, because mineral oil is a non-biodegradable compound.

To solve these inconveniences, in the present invention, a coating film was applied, such as a silicone film (see FIG. 3).

For the present invention, it was determined that the compound to be used for the coating of the porous surface of the activated carbon and to produce the second layer of the multilayer filter-adsorbent article, should be an inert and biodegradable polymer such as liquid silicone, specifically dimethylpolysiloxane.

As mentioned above, the molecular structure of liquid silicone comprises methyl groups (neutral charge) and an oxygen group (polar charge), both linked by silica atoms (an electrophilic), which enables them to form a chain. This configuration allows non-polar bonds to form in the hydrophobic zones of the surface of the activated carbon using the methyl groups. On the other hand, when positively charged functional groups are present on the surface of the activated carbon, they interact through polar bonds with the oxygen group of the silicone. Finally, the presence of negatively charged functional groups on the surface of the activated carbon, interact with the silica atom, due to their electrophilic character by the displacement of the electron cloud by the oxygen molecule. All these interactions contribute to generate a “silicon film” that covers the functional groups present in the pore of the activated carbon and, consequently, prevent direct contact with the active agent, such as a permanganate salt.

To obtain this second layer of the multilayer filter-adsorbent article, an aqueous mixture (mixture A) is prepared, which in the present invention is put in contact with the porous support activated carbon as will be detailed below, in such a way as to achieve a high-performance molecular lamination.

In a first stage, a silicone film is obtained which, when adhered to the surface of the activated carbon by means of apolar bonds (methyl group), on the opposite side, leaves the oxygen group available or exposed. The oxygen group (negative polarity) interacts with the manganese atom (positive polarity) and thus, the permanganate salt anchors to the silicone (see FIG. 4).

To obtain this third layer of the multilayer filter-adsorbent article, an aqueous mixture (mixture B) is prepared, which in the present invention is put in contact with the porous support activated carbon containing the second layer of silicone, as explained in the procedure for achieving a high-performance molecular lamination

In this second stage, a surface covered with the permanganate salt (MnO—) is obtained, with its active site (—MnO—) available to the medium, that is, a functional product with oxidative properties to act on the compounds (chemical structures and VOCs) responsible for the offensive odors produced in the breeding grounds of animals such as pigs and birds.

The process to obtain the multilayer filter-absorbent article comprises three stages: coating the porous support corresponding to activated carbon (first layer) with a mixture (A), containing water, silicone (dimethylpolysiloxane) and low foam surfactants (ethoxylated alcohols), constituting the second layer; followed by coating with the mixture (B) prepared with Potassium permanganate and low foam surfactant (ethoxylated alcohol) in an acidic aqueous medium, thus forming a third layer. Finally, a drying process is carried out by eliminating the excess water contained in the pores and thus, obtaining the product of the present invention, the multilayer filter-adsorbent article.

Although, in the state of the art an impregnation procedure under vacuum conditions is described as in the present invention (with some adaptations), the purpose has been to seal surfaces of articles which are exposed to extreme conditions, and this procedure has not been applied, as described in the present invention, on surfaces of high porosity and large surface area, with the aim of creating a multilayer filter-adsorbent article of large surface area and functionality.

The process flows used are described below (FIG. 5).

To begin the molecular lamination procedure of the multilayer filter-adsorbent article of the present invention, first, mixtures (A) and (B) are prepared.

As shown in FIG. 5, the watery mixture A (3) is prepared in the mixing tank (2). The mixture of the present invention has the advantage over the state of the art of other mixtures because it contains a low foaming surfactant agent (ethoxylated alcohol) which makes it possible to lower the surface tension of the mixture, facilitating and enabling the penetration of the solutes into the pores of the activated carbon. In this way the silicone molecules contained in the mixture (A) are distributed uniformly on the surface of the pore walls of the activated carbon, forming the second layer.

To determine a coating value with the mixture A, in practice, the chemical agent was considered at a low concentration (less than 10% w/v) and of surfactant (less than 2% w/v) in weight/volume of the total mixture.

Mixture A is transferred (4) to the degassing tank (5), where a vacuum of less than 10,000 Pa is applied to it for 5 to 10 minutes by means of a vacuum pump (6), in order to produce a degassing (7), preventing the gases dissolved in the mixture from interfering with the breaking of the surface tension in the molecular lamination stage of the pores of the activated carbon.

At the same time, the porous support filter-adsorbent multi-layer article prior to the invention, contained in a basket (8) is introduced to control the buoyancy, in the autoclave (10), where a vacuum (11) lower than 200 Pa is applied for a period of between 20 and 30 minutes, giving the porous support the distinctive property of not containing air in the pores. A vacuum pump (6) is used to achieve this level of vacuum.

The next stage is started by transferring (12) mixture A from the degassing tank (5) to the autoclave (10), with the essential characteristic that this filling must be done in an ascending way. Likewise, during the entire process of upward filling (12) of the autoclave (10), it is a fundamental condition to maintain a vacuum level below 500 Pa, since only in this way is it possible to quickly access the interior of the pores of the activated carbon while these are free of oxygen.

In this way, it is ensured that the molecular lamination, which consists of the molecules being uniformly distributed throughout the porous support, constitutes a homogeneous and consistent layer over time. Then, the filter-adsorbent article is kept submerged and under vacuum for a period of 20 to 30 minutes.

Afterwards, the vacuum is broken (15). and the autoclave is unloaded (10), transferring the mixture (16) back to the degassing tank (5), using the remaining vacuum of the degassing tank. Once the autoclave (10) is emptied, the machinery is cleaned.

In the next stage of the procedure, the basket (8) with the filter-adsorbent article inside it is removed from the autoclave (10) and transferred (18) to a centrifuge (20). A key (17) is used for this function. Then a centrifugation stage (21) is applied between 200 and 1000 rpm for a period of 1 to 6 minutes at a gradual upward speed, to eliminate the excess of mixture.

Finally, the filter-adsorbent article with the second layer, already centrifuged, is transferred (22) again to the autoclave (10), where it is subjected to a vacuum below 500 Pa (23) and a temperature between 30° C. and 36° C. (24).

The combination of both factors, vacuum and temperature, allows a rapid evaporation of the water at low temperature, achieving that the silicone (dimethylpolysiloxane) is deposited on the porous surface and forms a uniform layer. This corresponds to the last stage of the process of molecular lamination of the second layer, remaining only to remove the basket (8) with the porous support contained in its interior using the key (17).

In this way, a filter-adsorbent article containing silicone is obtained (the second layer), ready to continue with the procedure to add the third layer (permanganate salt).

For the second stage, mix B (3) is prepared in the mixing tank (2). The mixture contains a low foaming surfactant (ethoxylated alcohol) that allows the surface tension of the mixture to be lowered, facilitating the penetration of the solutes into the pores, while the presence of an acidic medium allows the stability of the permanganate salt. In this way the mixture is evenly distributed on the surface of the pore walls of the activated carbon.

To determine a coverage value with the solvent, in practice it was considered that the permanganate salt at a low concentration (less than 6% w/v) and of a surfactant (less than 1% w/v) in weight/volume of the total mixture.

Mixture B is transferred (4) to the degassing tank (5), where a vacuum of more than 10,000 Pa is applied for 5 to 10 minutes by means of a vacuum pump (6), in order to produce a degassing (7), preventing the gases dissolved in the mixture from interfering with the breaking of the surface tension in the molecular lamination stage of the pores of the activated carbon.

At the same time, (9) the filter-adsorbent article with the silicone layer, contained in a basket (8), is introduced into the autoclave (10), where it is applied in a vacuum (11) below 200 Pa, for a period of 20 to 30 minutes, giving the filter-adsorbent article with the silicone layer the distinctive property of not containing air in the pores. A vacuum pump (6) is used to achieve this level of vacuum.

The next stage is started by transferring (12) the mixture from the degassing tank (5) to the autoclave (10), with the essential characteristic that this filling must be done in an ascending way. Likewise, during the entire process of upward filling (12) of the autoclave (10), it is a fundamental condition to maintain a vacuum level below 500 Pa, since only in this way is it possible to quickly access the interior of the pores of the activated carbon, while these are free of oxygen. In this way, the molecular lamination is ensured, which consists of the molecules being distributed uniformly throughout the filter-adsorbent article with the silicone layer, constituting a homogenous and consistent layer in the permanganate salt time. The filter-adsorbent article is maintained with the silicone and permanganate salt layers, submerged and under vacuum for a period of 20 to 30 minutes.

Then, the vacuum is broken (15) and the autoclave is unloaded (10), transferring the mixture (16) back to the degassing tank (5), taking advantage of the remaining vacuum in the degassing tank. Once the autoclave is emptied (10), the machinery is cleaned.

In the next stage of the procedure, the basket (8) with the filter-adsorbent article, with the layers of silicone and permanganate salt, contained inside, is removed from the autoclave (10) and transferred (18) to a centrifuge (20). A key (17) is used for this function. Then a centrifugation stage (21) is applied between 200 and 1000 rpm for a period of 1 to 6 minutes of gradual speed, to eliminate the excess of mixture.

Finally, the filter-adsorbent article with the layers of silicone and permanganate salt, already centrifuged, is transferred (22) again to the autoclave (10), where it is subjected to a vacuum below 500 Pa (23) and a temperature between 30° C. and 36° C. (24).

The combination of both factors, vacuum and temperature, allows a rapid evaporation of the water at low temperature, achieving that the permanganate salt is deposited on the porous surface and forms a uniform layer. This corresponds to the last stage of the molecular lamination process, remaining only to remove the basket (8) with the porous support contained in its interior using the key (17).

In the manner described here, the multilayer filter-adsorbent article of the present invention is finally obtained to eliminate offensive odors from the breeding of poultry animals, such as pigs and birds.

In order to determine the content of solids incorporated into the pores, the dry weight was measured by gravimetry and infrared radiation until a constant moisture-free mass was obtained.

In this way, the practice was developed and through a mass balance, the value of added mass of the multilayer filter-adsorbent article was obtained, where according to the experience previously described, values between 50% and 120% of added mass of silicone (second layer) in weight/dry; and between 200% and 300% of added mass of permanganate salt (third layer) in weight/dry, with respect to the weight of the activated carbon.

1-11. (canceled)

12. A multilayer filter-adsorbent article to continuously degrade the compounds responsible for offensive odors, said multilayer filter-adsorbent article comprising:
a first layer formed by a non-woven polyester fiber felt impregnated with activated carbon, forming a porous support;
a second layer formed by a film of dimethylpolysiloxane liquid silicone and low-foam surfactants (mixture A), wherein said second layer is arranged over a surface of the first layer in order to cover functional groups present in pores of the activated carbon; and
a third layer formed by a film comprising an active agent formed by a salt of potassium permanganate and a low-foam surfactant in an acidic aqueous medium (mixture B), said third layer being joined to a surface of the second layer, wherein an upper surface of said liquid silicone film of the second layer is joined to the active agent of the third layer by means of negative polarity bonds generated by an oxygen group of the silicone film and a lower surface of said liquid silicone film of the second layer is adhered to the activated carbon of the first layer by means of apolar bonds generated by a methyl group of the silicone film.

13. The multilayer filter-adsorbent article according to claim 12, wherein said low-foam surfactants are ethoxylated alcohols.

14. The multilayer filter-adsorbent article according to claim 12, wherein said non-woven polyester fiber felt is a non-woven polyethylene terephthalate (PET) fiber felt.

15. The multilayer filter-adsorbent article according to claim 14, wherein said non-woven polyethylene terephthalate (PET) fiber felt is impregnated with 40% activated vegetable carbon, having a specific surface area of at least 800 m²/g carbon and a pore volume of at least 0.15 m³/g carbon.

16. A process to obtain a multilayer filter-adsorbent article, said process comprising:
covering a first layer with a second layer, wherein said first layer comprises a porous support formed by a non-woven polyester fiber felt impregnated with activated carbon and said second layer comprises a mixture (A) of water, dimethylpolysiloxane and low foam surfactants;
covering said second layer with a third layer comprising a mixture (B) of a salt of potassium permanganate and a low foam surfactant in an acidic aqueous medium in order to obtain a multilayer filter-adsorbent article; and
drying said obtained multilayer filter-adsorbent article.

17. The process to obtain a multilayer filter-adsorbent article according to claim 16, wherein said low foam surfactants are ethoxylated alcohols.

18. A use of the multilayer filter-adsorbent article of claim 12, wherein said multilayer filter-adsorbent article is used to eliminate foul odors from barnyard animal husbandry.

19. The use of the multilayer filter-adsorbent article according to claim 18, wherein said multilayer filter-adsorbent article is used to eliminate foul odors from pigs and birds.
